(19) United States
(12) Patent Application Publication
(10) Pub. No.: US 2025/0035566 A1
(43) Pub. Date: Jan. 30, 2025

(54) APPARATUS FOR GAS IDENTIFICATION USING HIGH FREQUENCY MICROWAVE CAVITIES

(71) Applicant: ARAMCO SERVICES COMPANY, Houston, TX (US)

(72) Inventor: Jose Oliverio Alvarez, Houston, TX (US)

(73) Assignee: ARAMCO SERVICES COMPANY, Houston, TX (US)

(21) Appl. No.: 18/358,626

(22) Filed: Jul. 25, 2023

(51) Int. Cl. G01N 22/04; H01P 7/06; H01P 7/10

(52) U.S. Cl. CPC G01N 22/04; H01P 7/06; H01P 7/10

(57) ABSTRACT

Described is an apparatus for gas identification using a re-entrant microwave cavity. The re-entrant microwave cavity includes a tubular body having an exterior wall, a top portion, and a bottom portion. An inner conductor is positioned within the tubular body. A measurement conduit is positioned within the central conductor and extends from the bottom portion to the top portion of the tubular body. A cavity entry in connection with a first end of the measurement conduit is formed proximate the bottom portion of the tubular body to receive one or more gases from below the tubular body. A cavity exit in connection with a second end of the measurement conduit is formed proximate the top portion of the tubular body to release the one or more gases from the tubular body. A high frequency connector is configured to connect the tubular body to a microwave source.

### BACKGROUND

Natural gas is generally stored in depleted reservoirs, which are hydrocarbon reservoirs that have been depleted of recoverable products. Underground storage of hydrogen involves multiple injection and withdrawal cycles. Typically, a cushion gas, such as nitrogen gas or methane gas, is pre-injected into underground geological formations. Then, hydrogen is injected for temporary storage and withdrawal at a later time.

The purity of withdrawn hydrogen is important for effective energy generation and determination of hydrogen loss due to dissolution into reservoir hydrocarbons. Impurities in hydrogen may interfere with functioning of equipment used in the storage, distribution, and use of hydrogen fuel. For instance, trace oxygen and water may be produced when hydrogen is produced by electrolysis of water. Sulfur compounds from the feedstock may also be present in the hydrogen supply. The purity of the hydrogen may also be affected by microbial activity that creates methane or acetic acid. Accordingly, there exists a need for monitoring the purity of produced hydrogen gas.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to an apparatus for gas identification. The apparatus includes one or more re-entrant microwave cavities. Each re-entrant microwave cavity has a tubular body with an exterior wall, a top portion, and a bottom portion. An inner conductor is positioned within the tubular body, and a measurement conduit is positioned within the inner conductor, extending from the bottom portion to the top portion of the tubular body. A cavity entry is formed proximate the bottom portion and in connection with a first end of the measurement conduit. The cavity entry is formed to receive one or more gases from below the tubular body. A cavity exit is formed proximate the top portion of the tubular body and in connection with a second end of the measurement conduit. The cavity exit is formed to release the one or more gases from the tubular body. A high frequency connector attached with the exterior wall is configured to connect to a microwave source.

In another aspect, the measurement conduit is formed from a hydrogen-resistant material.

In another aspect, the measurement conduit is formed from a crystal material.

In another aspect, the measurement conduit is formed from a quartz material.

In another aspect, the apparatus further comprises a low-loss dielectric filling material between the exterior wall and the inner conductor.

In another aspect, the exterior wall is coated in a metal material.

In another aspect, the metal material is a Cobalt-Nickel-Vanadium alloy.

In another aspect, the cavity entry has a diameter in a range of 0.1 centimeters and 6 centimeters.

In another aspect, the microwave source is at least one single-port vector network analyzer.

In another aspect, the microwave source is at least one multi-port vector network analyzer.

In another aspect, the apparatus further comprises a plurality of hydrogen-resistant support structures attached with the bottom portion.

In another aspect, the plurality of hydrogen-resistant support structures are comprised of one or more of a fluoroelastomer, a perfluoroelastomer, and a nitrile rubber.

In one aspect, embodiments disclosed herein relate to a method for identifying a gas with one or more re-entrant microwave cavities. One or more re-entrant microwave cavities are positioned proximate a well. A produced gas is collected from the well within the measurement conduit of the one or more re-entrant microwave cavities. A microwave signal is applied to the produced gas. One or more properties of the produced gas is measured, and an analysis of the one or more properties is performed. A purity of the produced gas is determined from the analysis.

In another aspect, the produced gas is at least one of hydrogen, carbon dioxide, and hydrogen sulfide.

In another aspect, a plurality of re-entrant microwave cavities is arranged in an array proximate the well.

In another aspect, the microwave signal is applied with at least one single-port vector network analyzer.

In another aspect, the microwave signal is applied with at least one multi-port vector network analyzer.

In another aspect, the one or more re-entrant microwave cavities are positioned near a surface pipe proximate the well.

In another aspect, the one or more re-entrant microwave cavities are positioned downhole.

In another aspect, performing the analysis includes obtaining a complex permittivity of the produced gas.

Other aspects and advantages of the claimed subject matter will be apparent from the following description and the appended claims.

### BRIEF DESCRIPTION OF DRAWINGS

FIG. 1A illustrates a cross-sectional diagram of a re-entrant microwave resonator according to prior art.

FIG. 1B illustrates a top, perspective-view of the re-entrant microwave resonator of FIG. 1A according to prior art.

FIG. 2 illustrates a re-entrant microwave cavity according to one or more embodiments of the present disclosure.

FIG. 3 illustrates a 1 port Vector Network Analyzer (VNA) according to one or more embodiments of the present disclosure.

FIG. 4 illustrates a multi-port VNA according to one or more embodiments of the present disclosure.

FIG. 5 illustrates a computing system according to one or more embodiments of the present disclosure.

FIG. 6 illustrates an exemplary well site according to one or more embodiments of the present disclosure.

FIG. 7A illustrates an exemplary array for positioning re-entrant microwave cavities according to one or more embodiments of the present disclosure.

FIG. 7B illustrates an exemplary array for positioning re-entrant microwave cavities according to one or more embodiments of the present disclosure.

FIG. 8 illustrates a method for measuring the complex permittivity of gases according to one or more embodiments of the present disclosure.

### DETAILED DESCRIPTION

In one aspect, embodiments disclosed herein relate to an apparatus and method for measuring the complex permittivity of produced gases, such as hydrogen (H₂) gas, carbon dioxide (CO₂) gas, and hydrogen sulfide (H₂S) gas. Complex permittivity may be used to characterize a solid or a fluid. Moreover, at low frequencies (MHz up to low GHz) the real part of the complex permittivity is related to the density, and the imaginary part is related to the electrical conductivity. The higher the frequency, the closer the wavelength gets to the size of the molecules of the material, and more specific information about the composition may be obtained. Thus, at high enough frequencies, and with the proper measurement system, the complex permittivity may be used to identify or measure the purity of a produced gas.

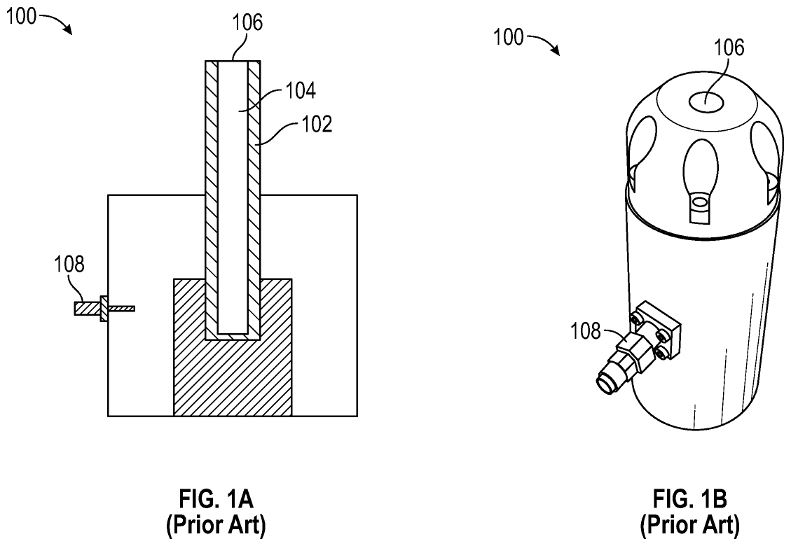

Microwave measurements provide values for the relative complex permittivity, or dielectric constant, of a material. Complex permittivity may be used to characterize properties of fluid, such as density and chemical composition. FIG. 1A is a sectional view of a prior art re-entrant microwave cavity (100) that may be utilized to determine complex permittivity of a liquid or a solid. The re-entrant microwave cavity (100) is a hollow, closed compartment comprised of a conducting material, such as a conductive metal. Radio frequency (RF) signals may be provided as input and output through input and output ports. Re-entrant microwave cavities may be used for oscillation filtering and amplification in the frequency range of approximately 3 megahertz (MHz) and approximately 300 MHz. The re-entrant microwave cavity (100) includes a sample vial (102). The re-entrant microwave cavity (100), like many re-entrant cavities, is designed for samples (104) of solids and liquids and operated in a batch-like process.

FIG. 1B illustrates a top, perspective-view of the re-entrant microwave cavity (100) of FIG. 1A. A cavity entry, in the form of an insertion hole (106) located centrally in a top wall of the re-entrant microwave cavity (100), permits samples to be introduced into the sample vial (102). The insertion hole (106) is under the cut-off frequency. The re-entrant microwave cavity (100) depicted in FIGS. 1A and 1B is sealed at its bottom. A coaxial line (108) is connected with a lateral wall of the re-entrant microwave cavity (100).

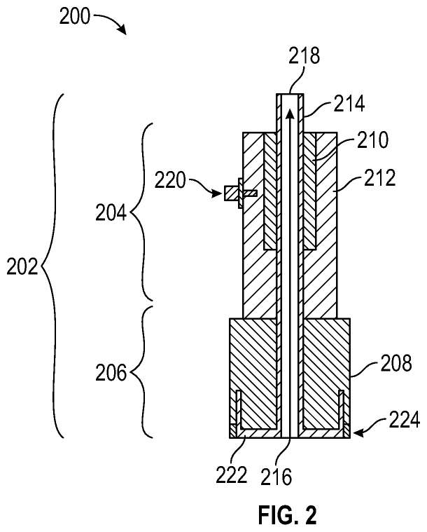

FIG. 2 illustrates a sectional view of a re-entrant microwave cavity (200) according to embodiments of the present disclosure. The re-entrant microwave cavity (200) is configured for measuring one or more properties of flowing gases or liquids which may be operated in a continuous process. The re-entrant microwave cavity (200) includes a cylindrical, tubular body (202) having a top portion (204) and a bottom portion (206). Although the size of the re-entrant microwave cavity (200) is related to the desired resonant frequency, in one or more embodiments, the diameter of the tubular body (202) may be between 0.1 centimeters (cm) and 6 cm. The length of the tubular body (202) may be any suitable length, such as between 0.5 cm and 12 cm. In further embodiments, the length of the tubular body (202) is at least double the diameter of the tubular body (202) to ensure that the resonant frequency is lower than the cutoff frequency of the re-entrant microwave cavity (200). The tubular body (202) may act as a microwave cavity allowing microwaves to bounce back and forth within the tubular body (202). At the resonant frequency of the tubular body (202), the microwaves reinforce to form standing waves within the tubular body (202). Within the tubular body (202) are a plurality of elements arranged as concentric layers, as described in detail below.

In one or more embodiments, an exterior wall (208) of the tubular body (202) may be formed from a hydrogen resistant protective coating to resist hydrogen embrittlement of the re-entrant microwave cavity (200). The coating may include a metal material, such as a Cobalt-Nickel-Vanadium (CoNiV) alloy, or any suitable hydrogen resistant composite. The coating may alternatively be formed from traditional metals, such as stainless steel or copper.

The re-entrant microwave cavity (200) further includes an inner conductor (210), having a first end, a second end, and a length extending between the first and second ends. In one or more embodiments, the length of the inner conductor (210) is between 0.3 cm and 6 cm. The inner conductor (210) is formed from one or more conductive materials, such as aluminum, copper, steel, and high-strength alloys. The inner conductor (210) may be coated with silver plating, nickel plating, or tin. Between the exterior wall (208) and the inner conductor (210) is a low-loss dielectric filling material (212), such as air, Teflon™, Rexolite®, glass, a crystal material, or a quartz material. A measurement conduit (214) passes through the filling material (212) and inner conductor (210). The measurement conduit (214) includes a first end, a second end, and a length extending between the first and second ends. As shown in FIG. 2, the measurement conduit (214) may extend the entire length of the tubular body (202). In one or more embodiments, the length of the measurement conduit (214) is between 0.5 cm and 12 cm, such as between 1.0 cm and 3 cm.

In one or more embodiments, both the inner conductor (210) and the measurement conduit (214) are hollow cylinders, and a diameter of the measurement conduit (214) is less than a diameter of the inner conductor (210). For instance, the diameter of the inner conductor (210) may be between 0.05 cm and 6.5 cm, and the diameter of the measurement conduit (214) may be between 0.03 cm and 6.3 cm, such as in a range of 0.1 cm and 6.0 cm. The measurement conduit (214) is formed to receive gases or liquids present below the tubular body (202). In one or more embodiments, the measurement conduit (214) is formed from a hydrogen-resistant material, such as crystal, quartz, and/or sapphire.

Unlike the prior art re-entrant microwave cavity (100) illustrated in FIGS. 1A and 1B, the re-entrant microwave cavity (200) includes a cavity entry (216), having a diameter, formed at a distal end of the bottom portion (206) of the tubular body (202) and a cavity exit (218) formed at a distal end of the top portion (204). The diameter of the cavity entry (216) may be between 1 millimeter (mm) and 2 cm. In one or more embodiments, the cavity entry (216) is connected with (or continuous with) a first end of the measurement conduit (214) proximate the bottom portion (206) of the tubular body (202). In another embodiment, the cavity entry (216) is substantially funnel-shaped, such that the diameter of the cavity entry (216) gradually increases as it extends from the bottom portion (206) in a direction of the cavity exit (218) proximate the top portion (204). In one or more embodiments, the cavity exit (218) is formed proximate the top portion (204) in connection with (or continuous with) a second end of the measurement conduit (214). The cavity exit (218) is configured to release the one or more gases that enter the tubular body (202).

Since the re-entrant microwave cavity (200) is not sealed at both ends, gas is permitted to flow through the cavity entry (216) and through the measurement conduit (214), exiting the cavity exit (218). The cavity entry (216) has a diameter that depends on the radio frequency range needed for optimal sample identification. For example, hydrogen (H₂), carbon dioxide (CO₂), and hydrogen sulfide (H₂S) may require frequencies in the range of approximately 20 gigahertz (GHz) and approximately 500 GHz for identification. The re-entrant microwave cavity (200) may further comprise an input port in the form of a high frequency connector (220) connected with the exterior wall (208) of the tubular body (202) via a fastening mechanism (e.g., screw thread, braces, bayonet mount). The high frequency connector (220) is configured to connect to a source of microwaves. In one or more embodiments, the high frequency connector (220) is a connector having a size of 1 mm or less.

The hydrogen-resistant material of the measurement conduit (214) may extend past the cavity entry (216) forming a hydrogen-resistant surface (222). The hydrogen-resistant surface (222) may be formed at the bottom portion (206) of the tubular body (202). In addition, support structures (224), such as O-rings, formed from a hydrogen-resistant material may be attached with at least a portion of the re-entrant microwave cavity (200). The support structures (224) may be formed of any suitable hydrogen-resistant material including, but not limited to, Viton™ fluoroelastomer, perfluoroelastomer, and nitrile rubber (or Buna-N). The support structures (224) may be configured to provide an attachment for the re-entrant microwave cavity (200) to a surface pipe, or other surface or structure, for stabilizing the re-entrant microwave cavity (200).

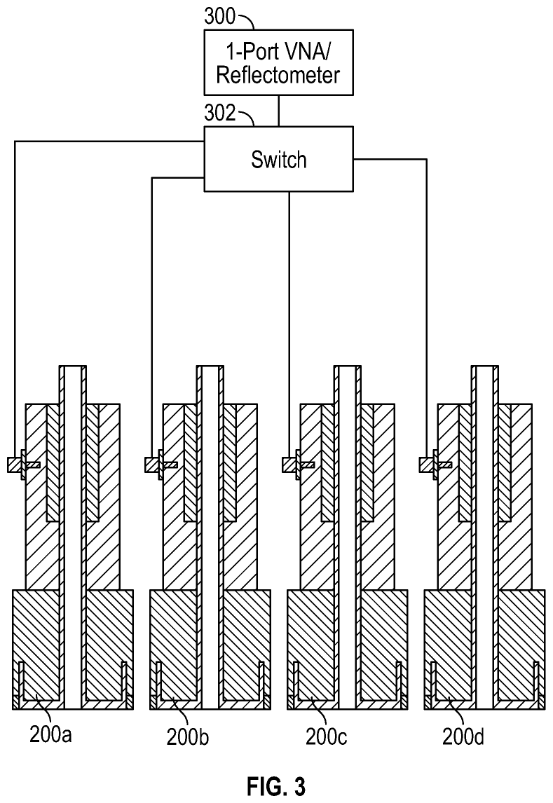

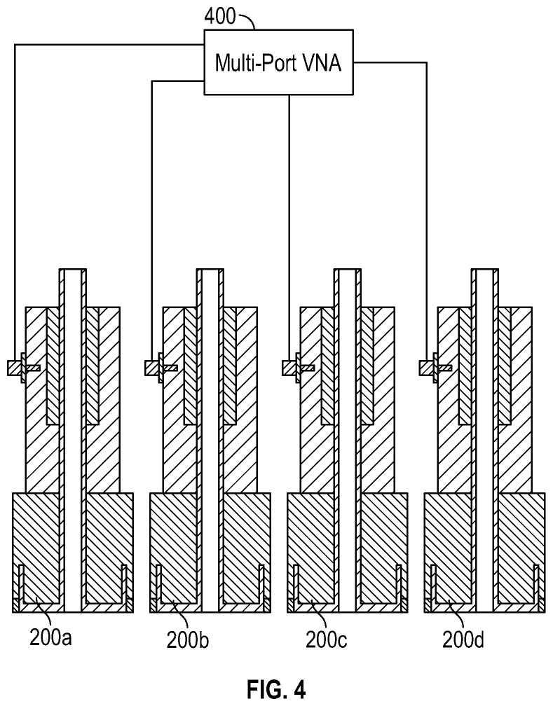

A single-port Vector Network Analyzer (VNA), also referred to as a reflectometer, or any other type of microwave source, may be used as a signal generator for generating the microwave emissions. A signal with a frequency of approximately 30 GHz to 500 GHz may be generated depending on the dimension of the cavity. As explained above, the frequency may be specific to the gases being analyzed. As shown in FIG. 3, the VNA may be a single-port VNA (300), also referred to as a reflectometer, or a series of single-port VNAs. The single-port VNA (300) may be connected with a switch system (302) configured to send a RF signal at a specific time for one or more specific re-entrant microwave cavities (200a, 200b, 200c, 200d). Alternatively, as depicted in FIG. 4, one or more multi-port VNAs (400) may provide the source of microwaves to the one or more re-entrant microwave cavities (200a, 200b, 200c, 200d). Multi-port VNAs (400) are configured to output multiple RF signals at once.

As a produced gas enters the measurement conduit (214), a reflected microwave signal may be received by the VNA (300 or 400). The VNA (300 or 400) may analyze the reflected microwave signal to determine a microwave reflection parameter of the produced gas. In one or more embodiments, the microwave reflection parameter is a S11 parameter. The S11 parameter represents how much power has been reflected from the produced gas. A property of the produced gas is determined from the microwave reflection parameter.

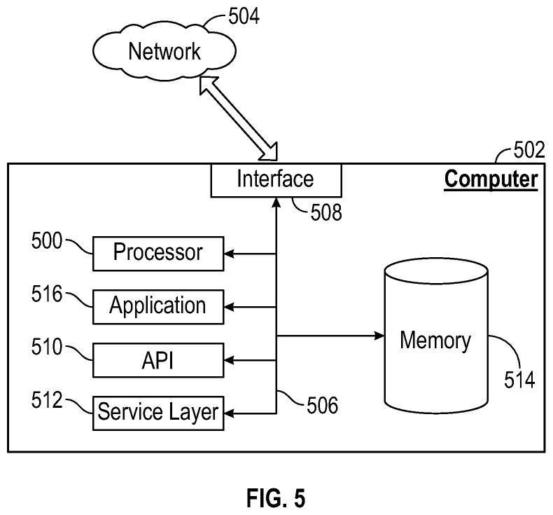

As shown in FIG. 5, a processor (500) of a computing system (502) in communication with the re-entrant microwave cavity may be configured to determine a property of the produced gas, such as complex permittivity, from the microwave reflection parameter. FIG. 5 depicts a block diagram of a computing system (502) used to provide computational functionalities associated with described algorithms, methods, functions, processes, flows, and procedures as described in this disclosure, according to one or more embodiments. The illustrated computing system (502) is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computing system (502) may include an input device, such as a keypad, keyboard, touch screen, or other device that can accept user information, and an output device that conveys information associated with the operation of the computing system (502), including digital data, visual, or audio information (or a combination of information), or a GUI.

The computing system (502) may serve in a role as a client, network component, a server, a database or other persistency, or any other component (or a combination of roles) of a computer system for performing the subject matter described in the instant disclosure. The illustrated computing system (502) is communicably coupled with a network (504). In some implementations, one or more components of the computing system (502) may be configured to operate within environments, including cloud-computing-based, local, global, or other environment (or a combination of environments).

At a high level, the computing system (502) is an electronic computing device operable to receive, transmit, process, store, or manage data and information associated with the described subject matter. According to some implementations, the computer system (502) may also include or be communicably coupled with an application server, e-mail server, web server, caching server, streaming data server, business intelligence (BI) server, or other server (or a combination of servers).

The computing system (502) may receive requests over network (504) from a client application (for example, executing on another computing system (502)) and responding to the received requests by processing the said requests in an appropriate software application. In addition, requests may also be sent to the computing system (502) from internal users (for example, from a command console or by other appropriate access method), external or third-parties, other automated applications, as well as any other appropriate entities, individuals, systems, or computers.

Each of the components of the computing system (502) can communicate using a system bus (506). In some implementations, any or all of the components of the computing system (502), both hardware or software (or a combination of hardware and software), may interface with each other or the interface (508) (or a combination of both) over the system bus (506) using an application programming interface (API) (510) or a service layer (512) (or a combination of the API (510) and service layer (512)). The API (510) may include specifications for routines, data structures, and object classes. The API (510) may be either computer-language independent or dependent and refer to a complete interface, a single function, or even a set of APIs. The service layer (512) provides software services to the computing system (502) or other components (whether or not illustrated) that are communicably coupled to the computing system (502). The functionality of the computing system (502) may be accessible for all service consumers using this service layer (512). Software services, such as those provided by the service layer (512), provide reusable, defined business functionalities through a defined interface. For example, the interface (508) may be software written in JAVA, C++, or other suitable language providing data in extensible markup language (XML) format or another suitable format. While illustrated as an integrated component of the computing system (502), alternative implementations may illustrate the API (510) or the service layer (512) as stand-alone components in relation to other components of the computing system (502) or other components (whether or not illustrated) that are communicably coupled to the computing system (502). Moreover, any or all parts of the API (510) or the service layer (512) may be implemented as child or sub-modules of another software module, enterprise application, or hardware module without departing from the scope of this disclosure.

The computing system (502) includes an interface (1706). Although illustrated as a single interface (508) in FIG. 5, two or more interfaces (508) may be used according to particular needs, desires, or particular implementations of the computing system (502). The interface (508) is used by the computing system (502) for communicating with other systems in a distributed environment that are connected to the network (504). Generally, the interface (508) includes logic encoded in software or hardware (or a combination of software and hardware) and operable to communicate with the network (504). More specifically, the interface (508) may include software supporting one or more communication protocols associated with communications such that the network (504) or interface's hardware is operable to communicate physical signals within and outside of the illustrated computing system (502).

The computing system (502) includes at least one computer processor (500). Although illustrated as a single computer processor (500) in FIG. 5, two or more processors may be used according to particular needs, desires, or particular implementations of the computing system (502). Generally, the computer processor (500) executes instructions and manipulates data to perform the operations of the computing system (502) and any algorithms, methods, functions, processes, flows, and procedures as described in the instant disclosure.

The computing system (502) also includes a memory (514) that holds data for the computing system (502) or other components (or a combination of both) that can be connected to the network (504). For example, memory (514) can be a database storing data consistent with this disclosure. Although illustrated as a single memory (514) in FIG. 5, two or more memories may be used according to particular needs, desires, or particular implementations of the computing system (502) and the described functionality. While memory (514) is illustrated as an integral component of the computing system (502), in alternative implementations, memory (514) can be external to the computing system (502).

The application (516) is an algorithmic software engine providing functionality according to particular needs, desires, or particular implementations of the computing system (502), particularly with respect to functionality described in this disclosure. For example, application (516) can serve as one or more components, modules, applications, etc. Further, although illustrated as a single application (516), the application (516) may be implemented as multiple applications (516) on the computing system (502). In addition, although illustrated as integral to the computing system (502), in alternative implementations, the application (516) can be external to the computing system (502).

There may be any number of computer systems associated with, or external to, a computer system containing computing system (502), wherein each computing system (502) communicates over network (504). Further, the term “client,” “user,” and other appropriate terminology may be used interchangeably as appropriate without departing from the scope of this disclosure. Moreover, this disclosure contemplates that many users may use one computing system (502), or that one user may use multiple computing systems (502).

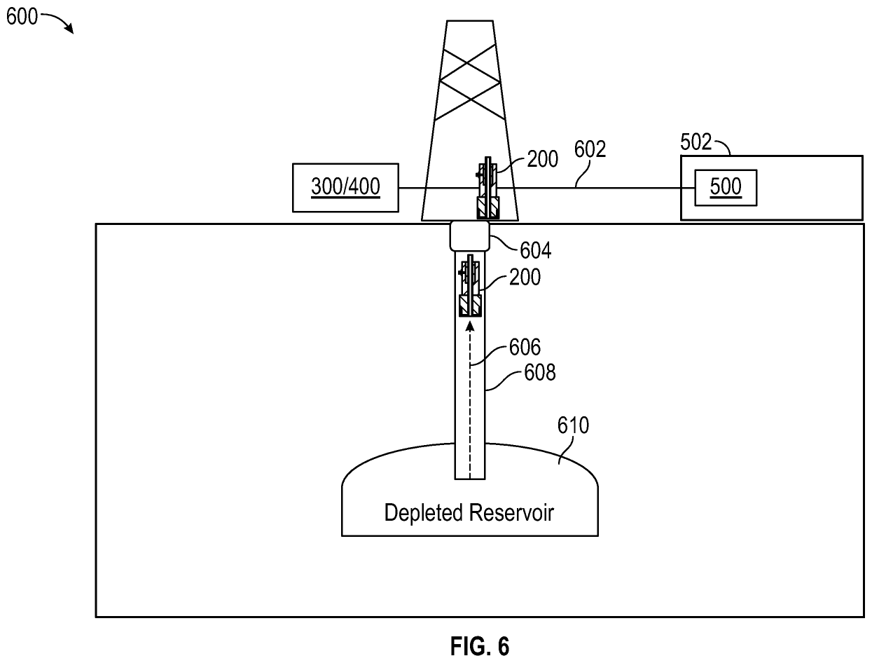

One or more of the components of the computing system (502) may be located at the surface of a well environment (600), as shown in FIG. 6. One or more re-entrant microwave cavities (200) may be connected to the processor (500) via a wired cable (602). The wired cable (602) may be electrically conductive such that information may be transferred between the processor (500) and the re-entrant microwave cavity (200). Alternatively, the connection may be wireless. Information may be transferred between the one or more re-entrant microwave cavities (200) and the processor (500) using any mechanism of information transfer, such as wireless information transfer. Through this connection, the processor (500) may instruct the VNA (300 or 400) to emit a microwave signal.

As mentioned above, data for each re-entrant microwave cavity (200) may be in the form of a microwave reflection parameter, or S11 parameter. Data may be sent to the processor (500) for inversion, and the complex permittivity values may be logged in real-time. A dielectric constant, the real part of the permittivity value, may be calculated from the magnitude of the microwave reflection parameter/S11 parameter. Specifically, when a microwave signal provided by the VNA (300 or 400) is applied to the produced gas (606), the polarization of molecules of the produced gas (606) is out of phase with the alternating current (AC) field. Accordingly, the measured complex permittivity includes real (ε′) and imaginary (ε″) parts that exhibit frequency dependence. E′ of the complex permittivity, also known as the dielectric constant, is a measure of the amount of energy from an external electrical field stored in the produced gas (606).

The VNA (300 or 400) may be used to measure the reflection coefficient (S11) of the microwave signal emitted into the re-entrant microwave cavity (200) for the measuring mode. The re-entrant microwave cavity (200) may have a resonant frequency in the range of, for example, 10 GHz to 300 GHz. Other frequencies, such as in the range of 300 GHz to 500 GHz, may be used without departing from the disclosure. The VNA (300 or 400) may further include a receiver to measure the microwave signal in the re-entrant microwave cavity (200). Comparison of the received microwave signal with the emitted microwave signal allows determination of ε.

The loss factor of the complex permittivity, ε″, quantifies the ability of the produced gas (606) to dissipate the absorbed energy of the external electrical field (e.g., by converting it into heat). The loss factor is zero for lossless materials. At a given frequency, ε″, leads to absorption loss if the value is positive and absorption gain if the value is negative. With different substances having different ε″ versus temperature characteristics, presence of different substances may be determined based on an assessment of ε″ over a temperature range. The lower the losses, the more conductive material is needed in, for instance, an outer wall of the inner conductor (210). The complex permittivity of the produced gas (606) may, thus, be obtained in, for example, silver-coated walls, which will permit classifying the produced gas (606) according to its complex-valued permittivity ε.

Using a resonance-based method, the complex permittivity may be determined by first measuring the resonant frequency and quality factor (q-factor) of the empty re-entrant microwave cavity (200) (in absence of the produced gas (606)), followed by measurements when the produced gas (606) is present. This method measures the spectrum of the reflection coefficient S11 where the resonant frequencies are given by specific troughs in the spectra. The Q-factor is obtained from the loaded empty re-entrant microwave cavity (200), and it depends on the resonant frequency, total electromagnetic energy in the cavity, and the total power dissipated by the cavity. The complex permittivity of the produced gas (606), such as hydrogen, may be computed using the frequency and q-factor. The complex permittivity value that is associated with the gas of interest (e.g., hydrogen) may be used as a baseline, and any deviation from that value beyond a predetermined threshold may indicate the presence of impurities. Machine learning algorithms may be used to train the computing system (502) on the values of the possible gas mixture. The processor (500) may be connected to a monitor that may be used to monitor and alert to the changes in the microwave reflection parameters/S11 parameters and changes in complex permittivity values.

As depicted in FIG. 6, one or more re-entrant microwave cavities (200) may be formed to be placed on a surface pipe (604), or below surface (i.e., wired downhole), to receive produced gases (606) from the well (608). The produced gas (606) may be recovered from a depleted reservoir (610) of an oil and gas field and then captured with the re-entrant microwave cavity (200). The measurement of complex permittivity occurs slightly above an internal metal/composite boundary of the re-entrant microwave cavity (200). Therefore, the open bottom portion of the re-entrant microwave cavity (200) does not affect the complex permittivity measurement.

In one or more embodiments, multiple re-entrant microwave cavities (200) may be arranged in an array, such as a circular array, proximate the well (608). Since the re-entrant microwave cavities (200) are intended for measuring the purity of gases, such as hydrogen, high microwave frequencies and smaller cavities are needed. A single re-entrant microwave cavity may choke the flow of gas. Multiple re-entrant microwave cavities are needed to account for the entirety of the gas flow. Therefore, an array of re-entrant microwave cavities (200) may be implemented.

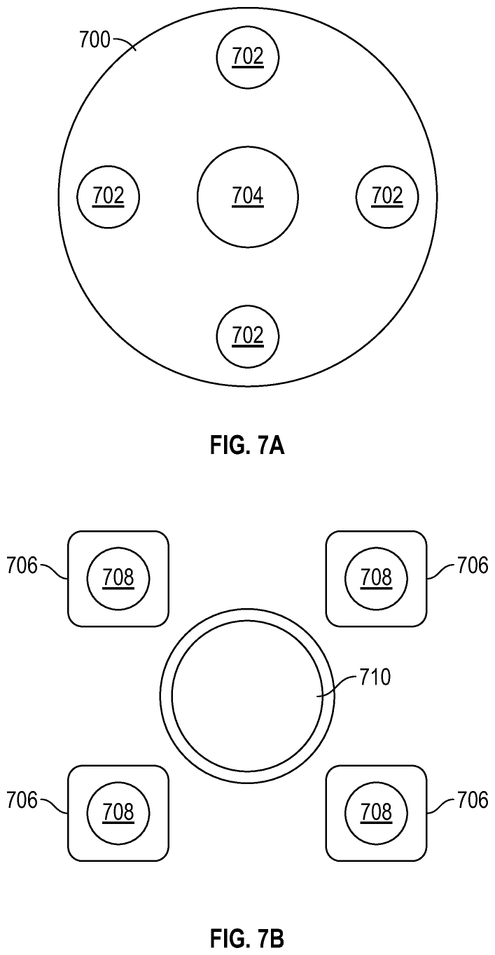

FIGS. 7A and 7B illustrate top views of exemplary arrays for positioning of re-entrant microwave cavities. In an embodiment shown in FIG. 7A, one or more re-entrant microwave cavities may be connected to a structure formed from a hydrogen-resistant material. The structure may be a circular plate (700) that includes multiple holes (702), each formed to receive a single re-entrant microwave cavity. Each re-entrant microwave cavity may be inserted into a hole (702) and secured to the circular plate (700) via one or more fastening elements, such as screws, bolts, or the like. In addition, the circular plate (700) may include a center opening (704) sized to surround the opening of a surface pipe. Alternatively, each re-entrant microwave cavity may be connected to a separate hydrogen-resistant structure (706) having an opening (708) therein, as shown in FIG. 7B. Each hydrogen-resistant structure (706) and connected re-entrant microwave cavity (200) may then be arranged proximate the opening (710) of a surface pipe in any desired arrangement or array.

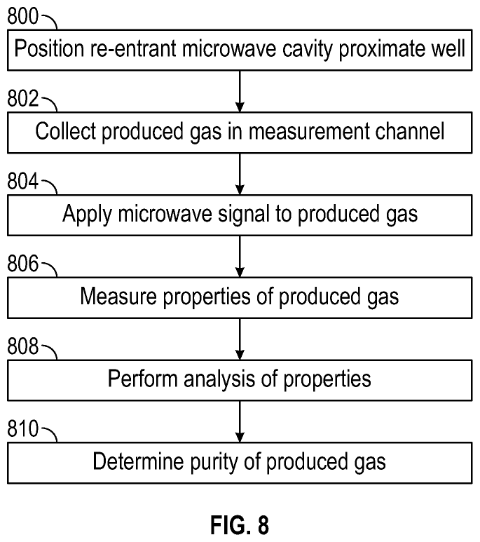

FIG. 8 illustrates a method for identifying a gas with one or more re-entrant microwave cavities according to embodiments of the present disclosure. In a first block (800), one or more re-entrant microwave cavities are positioned proximate a well. In a second block (802), at least one produced gas from the well is collected within the measurement channel of the one or more re-entrant microwave cavities. In a third block (804), a microwave signal is applied to the at least one produced gas. One or more properties of the at least one produced gas is measured in a fourth block (806). In a fifth block (808), an analysis of the one or more properties is performed. In a sixth block (810), a purity of the at least one produced gas is determined from the analysis.

Embodiments of the present disclosure may provide at least one of the following advantages. Previous re-entrant cavity models are designed for solids and liquids inside a vial. The configuration of the re-entrant microwave cavity according to embodiments of the present disclosure permits all flowing gases, including hydrogen, to be analyzed and monitored without constant calibration.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. An apparatus for gas identification, comprising:
one or more re-entrant microwave cavities, wherein each re-entrant microwave cavity comprises:
a tubular body having an exterior wall, a top portion, and a bottom portion;
an inner conductor positioned within the tubular body;
a measurement conduit positioned within the inner conductor and extending from the bottom portion to the top portion of the tubular body;
a cavity entry formed proximate the bottom portion, wherein the cavity entry is in connection with a first end of the measurement conduit and formed to receive one or more gases from below the tubular body;
a cavity exit formed proximate the top portion, wherein the cavity exit is in connection with a second end of the measurement conduit and formed to release the one or more gases from the tubular body; and
a high frequency connector attached with the exterior wall and configured to connect to a microwave source.

2. The apparatus of claim 1, wherein the measurement conduit is formed from a hydrogen-resistant material.

3. The apparatus of claim 2, wherein the measurement conduit is formed from a crystal material.

4. The apparatus of claim 2, wherein the measurement conduit is formed from a quartz material.

5. The apparatus of claim 1, further comprising a low-loss dielectric filling material between the exterior wall and the inner conductor.

6. The apparatus of claim 1, wherein the exterior wall is coated in a metal material.

7. The apparatus of claim 6, wherein the metal material is a Cobalt-Nickel-Vanadium alloy.

8. The apparatus of claim 1, wherein the cavity entry has a diameter in a range of 0.1 centimeters and 6 centimeters.

9. The apparatus of claim 1, wherein the microwave source is at least one single-port vector network analyzer.

10. The apparatus of claim 1, wherein the microwave source is at least one multi-port vector network analyzer.

11. The apparatus of claim 1, further comprising a plurality of hydrogen-resistant support structures attached with the bottom portion.

12. The apparatus of claim 11, wherein the plurality of hydrogen-resistant support structures are comprised of one or more of a fluoroelastomer, a perfluoroelastomer, and a nitrile rubber.

13. A method for identifying a gas with the one or more re-entrant microwave cavities of claim 1, comprising:
positioning the one or more re-entrant microwave cavities proximate a well;
collecting at least one produced gas from the well within the measurement conduit of the one or more re-entrant microwave cavities;
applying a microwave signal to the at least one produced gas;
measuring one or more properties of the at least one produced gas;
performing an analysis of the one or more properties; and
determining a purity of the at least one produced gas from the analysis.

14. The method of claim 13, wherein the at least one produced gas is at least one of hydrogen, carbon dioxide, and hydrogen sulfide.

15. The method of claim 13, wherein positioning the one or more re-entrant microwave cavities comprises arranging a plurality of re-entrant microwave cavities in an array proximate the well.

16. The method of claim 13, wherein the microwave signal is applied with at least one single-port vector network analyzer.

17. The method of claim 13, wherein the microwave signal is applied with at least one multi-port vector network analyzer.

18. The method of claim 13, wherein the one or more re-entrant microwave cavities are positioned near a surface pipe proximate the well.

19. The method of claim 13, wherein the one or more re-entrant microwave cavities are positioned downhole.

20. The method of claim 13, wherein performing the analysis further comprises obtaining a complex permittivity of the at least one produced gas.
